(19) United States
(12) Patent Application Publication
(10) Pub. No.: US 2012/0137977 A1
(43) Pub. Date: Jun. 7, 2012

(54) Food Production System From Biomass With Heat And Nutrient Recovery

(75) Inventors: Nicholas Hermes, Vancouver (CA); James Alexander Sawada, Edmonton (CA)

(73) Assignee: URBAN STREAM INNOVATION INC., Vancouver (CA)

(21) Appl. No.: 13/304,645

(22) Filed: Nov. 26, 2011

Related U.S. Application Data
(60) Provisional application No. 61/419,250, filed on Dec. 3, 2010.

(51) Int. Cl. A01K 67/033; A01K 67/00

(52) U.S. Cl. 119/6.5; 119/6.7

(57) ABSTRACT

A food production system having improved heat and nutrient recovery is disclosed. The system employs waste biomass and comprises a composter that partially composts the waste biomass, an invertebrate (e.g. worms) culture unit, a delivery subsystem to deliver partially composted waste biomass from the composter to the invertebrate culture unit in a temperature range suitable for the invertebrate culture, a food (e.g. fish or crustaceans) culture unit for producing the food operating in a temperature range suitable for the food culture, a delivery subsystem to deliver invertebrates from the invertebrate culture unit to the food culture unit, and a heat exchange subsystem providing for exchange of heat from the composter to the food culture unit. The subsystem comprises a controller for controlling the exchange of heat such that the food culture unit is maintained in the temperature range selected for the food culture.

### CROSS REFERENCE TO RELATED APPLICATIONS

This application claims benefit under 35 U.S.C. 119(e) of U.S. Provisional Patent Application Serial No. 61/419,250, filed Dec. 3, 2010 and entitled “Food Production System From Biomass With Heat And Nutrient Recovery” which is incorporated herein by reference in its entirety.

### FIELD OF THE INVENTION

The present invention relates to systems for producing food in which composting of biomass is involved. In particular, it relates to systems which efficiently recover heat and nutrients from the processes involved therein.

### BACKGROUND

Modern agricultural practices require large inputs from non-renewable resources including oil, nitrogen, and phosphorus to support production. Western agricultural practices are typically monoculture—a single species grown in isolation and the imbalances in such systems are typically balanced by adding fertilizers, pesticides and other petrochemical derivatives to make up deficits in such an unnatural system. As the requirement for food production increases and the cost of energy climbs in combination with the growing scarcity of the raw materials needed to sustain current agricultural practices, the need to recycle energy and nutrients will grow in step with these trends.

The farming of aquatic species on land or in the water (collectively termed aquaculture) is a growing industry because the natural ecosystems cannot keep up with the rate at which humanity harvests. We are at a time when the global consumption of fish and other aquatic species is the highest it's ever been and yet wild fish stocks are at an all time low and many species are facing extinction. Early developments saw aquaculture employ open-net farms, proving it is possible to raise most type of aquatic species in confinement. Such practices provide feed to the fish to augment what they can obtain naturally and use an existing body of water to dilute and eliminate the wastes generated by the fish. There is increasing pressure to have the industry shift to ‘closed-containment’ methods; particularly land-based aquaculture. Closed-containment practices require the operator to control the environment and provide a rich feed source. Closed-containment operations also have a requirement of treating the fish wastes before discharging the stream or recycling it back to the tanks. These requirements substantially increase the costs associated with closed-containment aquaculture.

As cities grow and agricultural reserves are converted into housing, our ability to generate food in close proximity to its use is being challenged. These trends result in food being transported even further distances to compensate for the expansion of urban centers. Not only are increasing amounts of fossil fuels being used to move food longer distances, the food waste generated may have to travel longer distances to reach processing or landfill facilities outside of the city.

The problems with disposing of the biomass extend beyond transportation costs associated with it. Incineration of wet, biodegradable biomass is fuel intensive and leaves nothing useful behind. Land-filling the biomass can recapture some of the energy value of the biomass by collecting the methane formed during anaerobic digestion, but all of the nutrient value in the biomass is lost. Composting the biomass allows for its nutrients to be reused but, normally, the energy value in the material is lost in the conversion.

The need is growing to re-engineer food production systems that do more with less by reusing and recycling energy and nutrients. Such systems will be fundamentally more sustainable because the requirement for external inputs will be greatly reduced. The reduction in external inputs will reduce the cost of operation and enhance the security of the food supply. Sustainable food production systems need to focus on polyculture and even polytrophic systems because the balance created by combining biosystems mimics the synergies found in natural ecosystems.

By combining the benefits of energy and nutrient recovery from biomass with the intentional benefits derived from balanced, polyculture and polytrophic systems, the next stage in development of sustainable food production systems can be realized. Maximizing the recovery of energy and nutrients from waste materials and using them to produce food is not only environmentally sound but is economically favorable because the external inputs to the system are minimized.

In Sustainable Food Production using a combined Aquaponic and Vermicomposting: A Model for Waste-to-Resource Recycling, Nick Hermes (Honours undergraduate thesis for class CHBE 492, Dr. Anthony Lau professor, Department of Chemical Engineering, University of British Columbia, 2008), the author describes a system that could demonstrate an example of waste-to-resource recycling. The system involves an aquaponic system with tilapia fish in the aquaculture and a small selection of vegetable and herb plants (tomatoes, zucchini, spinach and basil). The feature allowing for sustainably producing food is the combining of it with an onsite worm-composting unit to produce worms (fish feed) and a solid fertilizer for the plants.

In U.S. Pat. No. 7,222,585, Jablonski discloses a method suitable for commercial aquaculture comprising at least partially filling a tank with an aqueous medium and adapting it to control the quality of the aqueous media by connection to a temperature control means, such as compost packed around the tank. It was suggested that the compost can also support worms or other living creatures, which may be used as food for the marine or freshwater organisms in the cells. However, no mention is made on how to manage the conflicts of maintaining thermophilic conditions in the compost while simultaneously managing a vermiculture system in the pile. For instance, the traditional practice of periodically mixing the compost pile to aerate the pile would harm the vermiculture operation by exposing the worms to the hot interior of the pile. No mention is made on how to harvest the worms from the compost pile without disturbing the stability of the thermophilic compost operation. And further, there is no description on how to maintain aerobic compost conditions for appropriate heat output or for controlling the temperature of the aquaculture tank to prevent for instance the compost pile from driving the water temperature beyond an acceptable temperature for the livestock.

Biosystem Solutions manufactures compost equipment which can generate aerobic compost and vermicompost products. Further, they suggest that that an aerobic compost process can be used to feed a vermicompost process. This configuration is beneficial for vermicompost because the majority of the thermophilic composting is complete yet a great deal of organic material remains to support the colonies of bacteria that feed the worms. However, the systems outlined have no capability of withdrawing heat from the thermophilic composting operation so all of the surplus heat from the composting process is lost to the environment. Similarly these systems are not designed to culture worms and have no capability of harvesting a portion of the worms. This situation is expected as conventional vermicompost operations rely on a relatively constant worm population fed with an abundance of biomass. In these configurations, the worms consume waste but the population does not grow. Such a configuration does not allow the worms to be used as a feed product in an aquaculture system because the worms taken out are not readily replenished in the system.

U.S. Pat. No. 7,135,332 describes a system in which a compost pile has heat recovered from it though a series of heat exchange elements. The heat is used to warm a conventional greenhouse by way of a heat distribution system. The system described uses a trench to contain the biomass and an auger-type device to move the biomass from one end of the channel to the other. Aeration is accomplished by paddles on the auger which turn the biomass over, ensuring that stagnant pockets do not develop. This type of compost management is a form of mechanical windrowing and is recognized to sacrifice a lot of heat to the environment. The particular form of heat exchange used in this design uses a phase-change heat pipe (Isobars®) which uses the latent heat of vaporization of water to move heat from a high temperature region of the pipe to a lower temperature region of the pipe. The efficiency of the heat transfer afforded by these phase-change heat exchange systems allows heat to be captured from within and above the compost pile. This is an important consideration in the design due to the quantity of heat lost during mixing. In the design, the Isobar® heat exchangers are used to move heat from the biomass to a water fluid reservoir from which the heated water is circulated through a conventional hot water distribution system to heat the air circulating through the greenhouse. The design of the system relies entirely on these thermosyphons and their placement above the biomass and in the walls of the retaining channel which contains the biomass. In no configurations however are the Isobars® configured to be in direct contact with the compost pile. Being located at the exterior of the pile also eliminates the opportunity to manage thermal gradients that can form within a compost pile.

The primary purpose of traditional compost units is to efficiently convert waste into carbon dioxide, heat and water, while minimizing the production of low-value residual solids and noxious gases. Conventional aerobic compost operations target the complete decomposition of the biomass in as short a period of time as possible. In conventional systems, the residence time may be on the order of 20 days or more. More sophisticated systems are emerging that may introduce a greater degree of mixing and/or a high degree of aeration to reduce the time required to completely decompose the material. In all cases, however, the end product is biomaterial suitable mainly as a soil remediation agent. This fully composted material lacks the necessary level of nutrients to sustain the bacterial colonies on which the worms in a vermiculture operation subsist. It is a misconception that worms eat organic waste. In fact, the worms eat the microbes that decompose organic waste in aerobic environments and so it is vital that the biomass used in a vermiculture operation contain sufficient nutrient value to sustain the lower-temperature, mesophilic bacteria while, at the same time, being depleted in nutrient value so as to not be able to sustain high temperature, thermophilic bacteria.

The present invention addresses the need for food production systems having improved heat and nutrient recovery. These and other benefits are provided as disclosed herein.

### SUMMARY OF THE INVENTION

A sustainable, balanced food production system employing waste biomass and efficient heat and nutrient recovery comprises a composter for partially composting the waste biomass, an invertebrate culture unit operating in a temperature range selected for the invertebrate culture, and a food culture unit for producing a food operating in a temperature range selected for the food culture. The food can be selected from the group consisting of vertebrates (e.g. fish) or arthropods (e.g. crustaceans). A delivery subsystem is provided to deliver partially composted waste biomass from the composter to the invertebrate culture unit in the temperature range selected for the invertebrate culture in order to support the invertebrate culture. A delivery subsystem is also provided to deliver invertebrates from the invertebrate culture unit to the food culture unit in order to support the food culture. And a heat exchange subsystem is provided for the exchange of heat from the composter to the food culture unit for heat recovery and for maintaining appropriate temperature control of the composting and the food culture. The subsystem comprises a controller for controlling the exchange of heat such that the food culture unit is maintained in the temperature range selected for the food culture.

In the system, the waste biomass is only partially composted, for instance for less than 5 days after the time when the peak composting temperature has been reached. In particular, the waste biomass may be composted for about 1 day after the time when the peak composting temperature has been reached.

The food production system is particularly suitable for aquaculture in which case the food culture unit is an aquaculture unit. A particularly suitable invertebrate culture unit for such a system is a vermiculture unit. In such a system, a suitable temperature range for the aquaculture is above ambient temperature and a suitable temperature range for the vermiculture is between about 15 to 25° C.

The controller may desirably control the temperature of at least a portion of the composter between about 50 to 70° C. However, so as not to be introducing undesirably warm material into the invertebrate culture unit, a cooling subsystem may additionally be provided between the composter and the invertebrate culture unit in order to cool the partially composted waste in that portion of the composter to be less than about 35° C. before delivering to the invertebrate culture unit.

The heat exchange subsystem may comprise a piping network and a heat exchange fluid (e.g. water from the aquaculture unit itself) within the piping network. The piping network may be arranged throughout the composter so as to reduce any thermal gradients within the compost.

The food production system lends itself to integrated vertical farming practices. For example, the composter can be located above the invertebrate culture unit and the delivery subsystem to deliver partially composted waste can therefore be assisted by gravity. Such a delivery subsystem can comprise a screw auger or a baffle. In a like manner, the invertebrate culture unit can be located above the food culture unit and the delivery subsystem to deliver invertebrates can also be assisted by gravity.

A fungi unit may optionally be included in the system. Waste solids from the invertebrate culture unit may be provided to the fungi unit, whereupon fungi is grown on the waste solids and thereafter a portion of the solids from the fungi unit is used in the composter. Such a fungi unit is coupled to the composter and to the invertebrate culture unit. A portion of the output of the invertebrate culture unit is provided as an input to the fungi unit and a portion of the output of the fungi unit is provided as an input to the composter. In addition, waste solids from the food culture unit can also be provided to the composter.

A photosynthesis unit may also optionally be included in the system. A nutrient solution using suspended solids, dissolved solids, soluble compounds and water from the food culture unit may be prepared which is then provided to the photosynthesis unit in order to grow photosynthetic organisms therein. Afterwards, a nutrient depleted solution from the photosynthesis unit may be provided to the food culture unit. Such a photosynthesis unit is coupled to the food culture unit. A portion of the output of the food culture unit is provided as an input to the photosynthesis unit and a portion of the output of the photosynthesis unit is provided as an input to the food culture unit. A portion of the output solids from the invertebrate unit may be provided as an input to the photosynthesis unit (e.g. as a slurry with the nutrient solution)

### BRIEF DESCRIPTION OF THE DRAWINGS

FIG. 1 depicts a schematic of the prior art food production system disclosed in the aforementioned thesis Sustainable Food Production using a combined Aquaponic and Vermicomposting System.

FIG. 2 shows a schematic of an exemplary comprehensive food production system comprising the basic system of FIG. 3.

FIG. 3 shows a schematic of a basic food production system comprising an appropriately interconnected composter, invertebrate and food culture units, and a heat exchange subsystem.

FIG. 4 shows a schematic of various components and inputs and outputs for an exemplary composter employed in the food production system.

### DETAILED DESCRIPTION

Certain terminology is used in the present description and is intended to be interpreted according to the definitions provided below. In addition, terms such as “a” and “comprises” are to be taken as open-ended. Further, all US patent publications and other references cited herein are intended to be incorporated by reference in their entirety.

Thermophilic is used to describe the environmental conditions, biological activity or zones of a biological system that are associated with high temperatures and the organisms that thrive there and/or produce heat. A thermophile is an organism and a type of extremophile that thrives at relatively high temperatures, between 45 and 80° C. [Madigan M T, Martino J M (2006). Brock Biology of Microorganisms (11th ed.). Pearson. pp. 136. ISBN 0-13-196893-9.]

Aerobic refers to an environment that is oxygenated and is used generally in this sense with a reference to organisms (aerobes) that can survive or thrive in the presence of oxygen. Different types of aerobes thrive in a wide range of oxygen concentration, with some preferring low levels and others requiring ambient levels. Therefore, even air with depleted oxygen is considered aerobic. While this most often refers to atmospheric oxygen levels (˜21% oxygen), the consumption of oxygen by the aerobes in compost and vermiculture could drop the concentration of oxygen. However as the oxygen level decreases, it increases the chances that facultative anaerobes could grow as well.

Anaerobic refers to the condition, biological activity or organisms “anaerobes” that are associated with an environment in the absence of oxygen. An anaerobe does not require oxygen and some anaerobes can not tolerate any oxygen. If biological activity is primarily conducted by anaerobes, the process can be referred to as anaerobic, even if there is trace oxygen present. Facultative anaerobes will even use oxygen if present. Anaerobic digestion would refer to the process of using anaerobic microbes in a bioreactor to turn organic waste into biogas and biosolids.

Aquaculture is the practice of growing aquatic organisms (such as fish farming) and also refers to the controlled environment that is used. It can be used as an all encompassing word for “aquaculture unit plus supplementary process steps, equipment, pre- and post-treatments etc”. A form of aquaculture is aquaponics, which incorporates hydroponics and aquaculture. In this sense the term, aquaculture is used a process step within the system especially in regard to water quality and biomass production.

Aquaponics is the practice and system that incorporates aquaculture and hydroponics.

Hydroponics is the practice of growing plants in a soil-less medium by providing a nutrient solution and supporting controlled environment. When incorporated in an aquaponic system, the phrase hydroponic unit may be used to refer to the process step where its nutrient uptake functions as water treatment for the system.

Mesophillic is used to describe ambient temperature ranges and the biological activity associated with this environment. Herein, beneficial mesophiles are present in each unit due to the temperatures involved. Mesophilic bacteria are those in which optimum growth generally occurs between 20 and 45° C., although they usually can survive and grow in temperatures between 10 and 50° C. Mesophiles are even present in low concentrations in ‘hot’ composting, providing the initial breakdown and heat generation to allow thermophiles to populate. Trace mesophiles may survive heating processes where small colder zones develop.

Composting is the aerobic decomposition of organic matter into humic materials, water, carbon dioxide and heat. It can refer to any biological processing of organic matter or waste that uses aerobic respiration. Compost may be used to refer to the final product of composting.

Vermiculture is the practice of raising invertebrates, for instance worms for production purposes. These may usually be red wigglers, eisenia foetida and similar species of compost and soil worms. Since the goal of vermiculture is to produce worms, reproduction is encouraged through feeding conditions, bedding material and proper stocking densities kept in check with regular worm harvesting.

Vermicompost is the practice of decomposing organic matter using compost worms. In this usage, the objective is mainly to compost material and/or produce castings, while the objective of vermiculture is mainly to produce worms. Although the species of worm is generally the same, different equipment and methodology may be used.

Nutrient refers to plant-available compounds and elements. When nutrients are dissolved in water, for use as a nutrient solution, the water is said to be undergoing nutrification. After plants have taken up some nutrients, it is then ‘depleted’ or ‘spent’ nutrient solution. The term can also be used to describe digestible compounds and elements present in feed materials, particularly the aquaculture feed matter.

Biomass refers to biological matter generally. In the present application, biomass will most often refer to dead/recently living, biological matter or biodegradable waste. ‘Living biomass’ is used herein when discussing the growth or living organisms and plants. ‘Waste biomass’ refers to biodegradable waste.

Biogas is produced by anaerobic digestion or fermentation of biodegradable materials (such as biomass, manure, sewage, municipal waste, green waste, plant material and energy crops) and may consist primarily of methane and carbon dioxide.

Culture refers to the practice of raising living species in general by providing the required inputs and environment for growth. For instance, composting is a microbial culture, raising worms is vermiculture, and farming aquatic species is aquaculture. Further, horticulture is the cultivation of plants, generally food crops and in soil medium, though hydroponics could be considered soil-less horticulture. It also refers to the science involved and control of proper growing environment

Biosolids generally refers to organic matter that has undergone treatment to reduce its Biological Oxygen Demand (BOD) and pathogen concentration. The term may be used to refer to the end-product of composting, vermicomposting, and anaerobic digestion.

Bioreactor refers to the system or process unit that performs a bio-chemical function by providing adequate growth conditions and inputs for specific species or group of organisms or plants.

Vermi, or worms, include compost worms or red wigglers. In this art, these terms generally refer to eisenia foetida and eisenia andrei, and occasionally Lumbricus rubellus.

Carbon to nitrogen ratio refers to the elemental balance between carbon and nitrogen in biodegradable waste or biomass. Compost microbes require a C:N ratio of about 25:1 to 30:1. This ratio ensures the microbes get enough energy from the carbon and nitrogen to sustain biological growth. As a material approaches the lower C:N ratio of 25:1 and below, it is referred to as nitrogen rich, which means it will be more likely to biodegrade quickly, requiring more oxygen to break down, increasing the reaction rate and ultimately the rate of heat generation. If insufficient oxygen is present, excess nitrogen could lead to odor formation. (Food waste, manure, fish waste, alfalfa, animal products, and grass clippings are typically nitrogen rich.) On the other hand, as a material approaches the upper C:N ratio of 30:1 and above, it is carbon rich and growth is then limited by the addition of nitrogen. At these ratios, decomposition is slower and cooler due to limited reaction rates. However, carbon rich composts rarely have associated odors. (Wood, straw, paper and cardboard, and dry leaves are typically carbon rich.)

Treated solids refers to partially degraded biological matter. Herein, it is biodegradable waste that has already undergone partial composting and also contains living microorganisms from the compost.

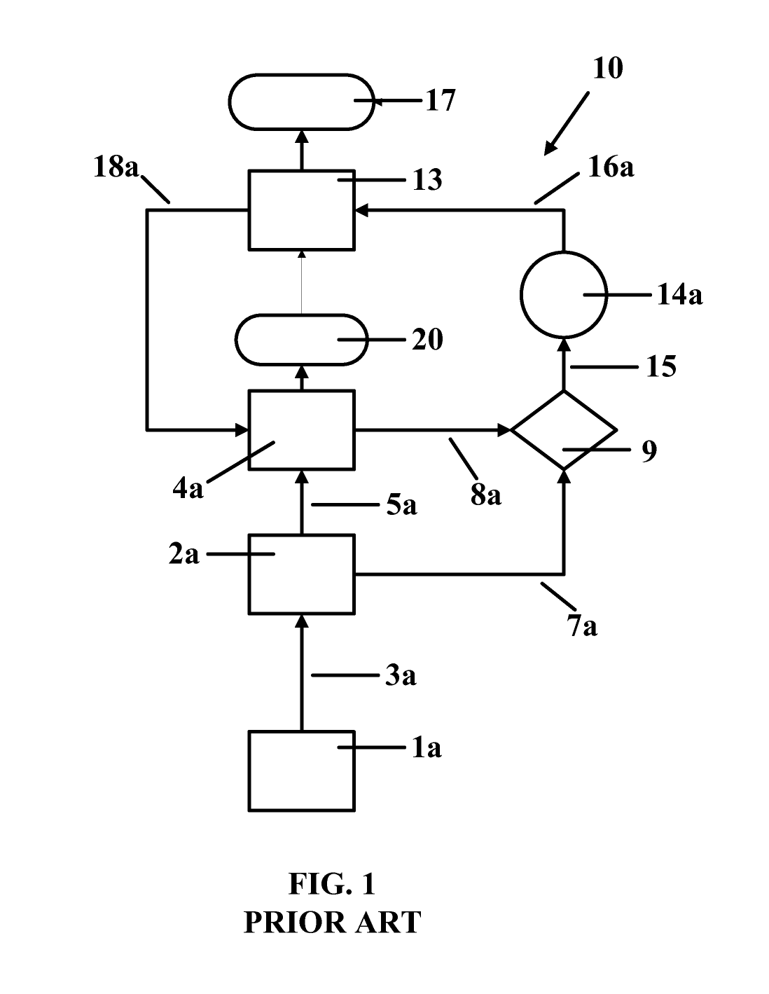

### FIG. 1:

FIG. 1 shows a schematic of the prior art food production system disclosed in the aforementioned thesis Sustainable Food Production using a combined Aquaponic and Vermicomposting System. As shown, prior art system 10 comprises an aerobic composter 1a, an invertebrate culture unit 2a (and specifically a vermiculture unit), and a food culture unit 4a (and specifically an aquaculture unit). Delivery subsystem 3a is provided to deliver composted material from composter 1a to vermiculture unit 2a. Delivery subsystem 5a is also provided to deliver cultured worms from vermiculture unit 2a to aquaculture unit 4a. The system however did not make use of heat produced during the aerobic composting.

Food such as freshwater fish or crustaceans are produced in aquaculture unit 4a and output therefrom as illustrated at 20. Prior art system 10 also comprises hydroponic unit 13 which obtains inputs originating from both vermiculture unit 2a and aquaculture unit 4a. Material, specifically worm castings, obtained from vermiculture unit 2a and return water plus waste solids obtained from aquaculture unit 4a are delivered by delivery subsystems 7a and 8a respectively to be combined as indicated at compost tea unit 9. The liquid extract from compost tea unit 9, i.e. compost tea, is delivered by delivery subsystems 15 to unit 14a where it is combined with nitrifying bacteria to produce nutrified return water (by extracting nutrients from the worm castings) which is subsequently delivered by delivery subsystem 16a to hydroponics unit 13. Plant product 17 is obtained from hydroponics unit 13 as shown and spent, reduced nutrient solution is returned via subsystem 18 to aquaculture unit 4a.

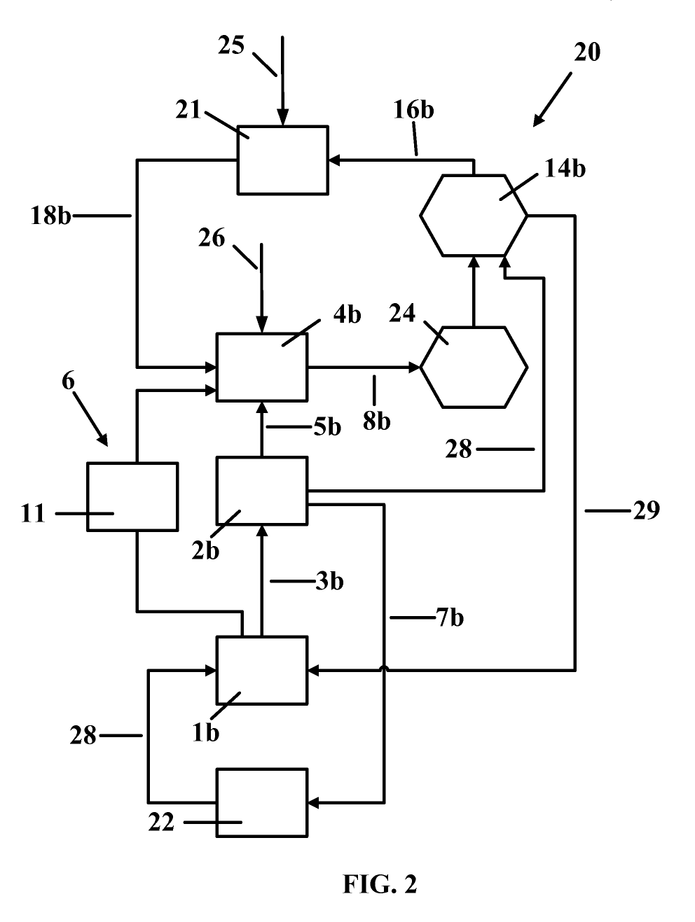

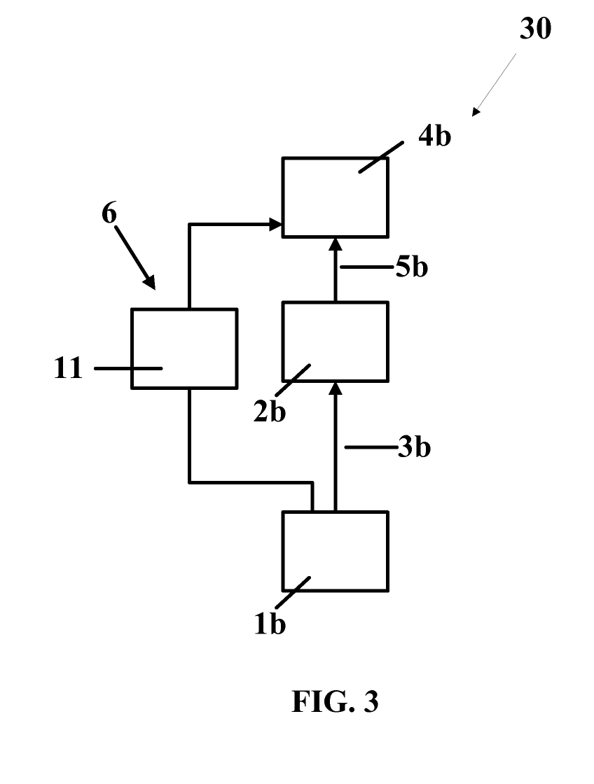

Schematics of exemplary food production systems of the invention appear in FIGS. 2 and 3. FIG. 3 shows a schematic of a basic embodiment 30 comprising an appropriately interconnected composter, invertebrate and food culture units, and a heat exchange subsystem. FIG. 2 shows a schematic of a more comprehensive, complex food production system 20 comprising the basic system 30 of FIG. 3 along with other useful subsystems.

### FIG. 3:

The basic embodiment shown in FIG. 3 offers both energy and nutrient recovery. System 30 comprises composter 1b, invertebrate culture unit 2b, food culture unit 4b, and heat exchange subsystem 6. Delivery subsystem 3b is provided to deliver partially composted waste biomass from composter 1b to invertebrate culture unit 2b. Delivery subsystem 5b is also provided to deliver cultured invertebrates from invertebrate culture unit 2b to food culture unit 4b. Heat exchange subsystem 6 is provided to exchange heat appropriately between composter 1b and food culture unit 4b. Heat exchange subsystem 6 comprises controller 11 for controlling this exchange of heat and also comprises other important structural elements that are discussed in more detail below. The combination of components in system 30 offsets the costs of operation for the production of food because the thermal requirements and nutrient requirements for food culture unit are provided for through the use of waste biomass.

Composter 1b shares some features with conventional composters in the art. However, composter 1b is unconventional in other aspects and particularly in that its function is to only partially compost the waste biomass provided to it. Composters generally utilize thermophylic bacteria to reduce waste biomass by means of a biological combustion process. The biological process is exothermic and, as a result, significant quantities of heat are produced during the combustion process. Typical conventional composters allow surplus heat to dissipate either passively or by purging the compost pile with air to remove surplus heat. Still others are insulated and aggressively mix or aerate the biomass within to reduce the time it takes to fully compost the material. In either case, the end product of conventional composters is a fully composted product which is useful mainly as a soil amendment. In a conventional composting system efforts may be made to maintain a target compost pile temperature but systems are not provided to allow for functionality to transfer heat from the decomposing biomass to another system.

Unconventional composter 1b however is designed to only partially compost waste biomass and also includes heat exchange elements so that it is possible to extract heat from the composting biomass and transport that heat to food culture unit 4b. The cost savings and ecological benefits associated with using waste heat to maintain an independent growing unit is significant. For instance, it has been calculated that an aerobic compost pile can provide 1.23 MJ/kg of total heat production (Physical management and interpretation of an environmentally controlled composting ecosystem—1992—Harper, Miller and Macauley). When the pile is operating at its highest temperatures, the biological combustion reaction rate is maximized and the compost pile is capable of generating 8.9 W/kg of biomass. Taking a heat capacity of water as 4.185 J/g K, it can then be estimated that 1 kg of composting biomass can raise the temperature of almost 300 L of water by 1 degree Celsius. If the potential heat of combustion from the biological process is captured and transferred to food culture unit 4b, a significant reduction in external heating demand can be realized.

A conventional composting system is not used for composter 1b because the product generated is too depleted in nutrients to sustain the bacterial culture necessary for the macro organisms in invertebrate culture unit 2b. As a result, composter 1b is designed to only partially decompose the waste biomass provided thereto. After spending 24 hours at the peak temperature in the thermophilic stage, most of the material within composter 1b will have reached the highest operating temperature but the bacteria will not have had time to break down all of the biomatter. The partially decomposed biomass is then conveyed via subsystem 3b to the invertebrate culture unit 2b. Preferably, the partially composted waste biomass should be unable to sustain temperatures above about 75° C. (when the majority of the organisms will die) and should be at a temperature between 55 and 75° C. In a plug-flow, top-fed system for instance, the material might be discharged after it has spent 1 day at peak temperature. Thus, if it is known that a given mass of material will reach peak temperature in 3 days (given the process conditions and heat recovery) then the residence time in composter 1b should be designed for about 4-5 days so that the partially treated biomass is discharged just after is has contributed to the peak thermophilic heat output.

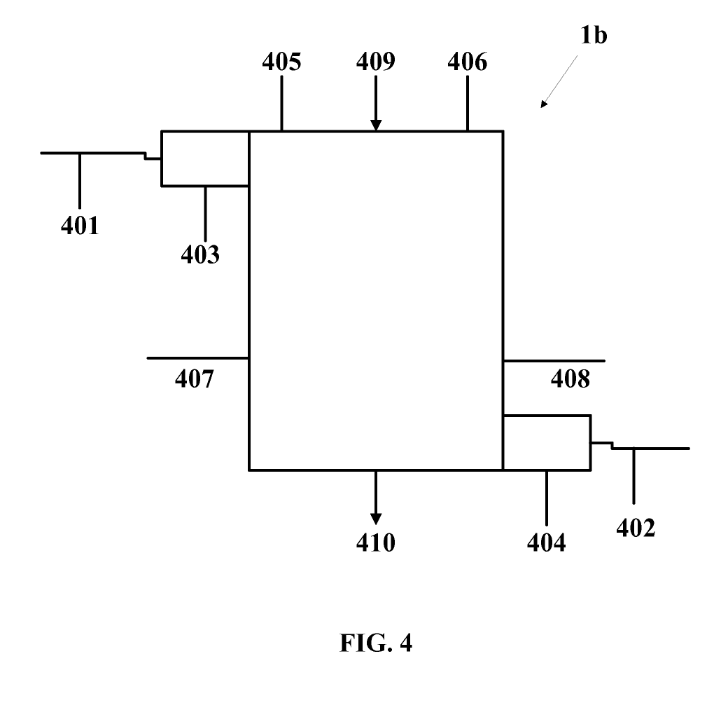

Composter 1b is comprised of a vessel for holding the biomass. The geometry of the compost vessel is not critical and examples of composters of various geometries are known in the art, e.g. circular, square, and rectangular geometries. Regardless of geometry, composter 1b will have access ports to allow biomass to be added thereto and to have composted material to be withdrawn therefrom. Typically the loading port is located at the top of the vessel and the withdrawal port is located at the bottom of the vessel. Further, composter 1b may be insulated to retain heat. Optionally, vents can be located at the top to facilitate the removal of moisture and heat. A more detailed schematic of composter 1b appears in FIG. 4 and is discussed in more detail below.

Composter 1b is filled with biomass which has the capacity to be acted upon by thermophilic bacteria. Such biomass may include, but is not limited to manure, food scraps, agricultural waste, landscaping waste, and waste water sludge. The types and ratios of each type of biomass may be selected to optimize the working conditions of composter 1b so as to maintain important properties such as moisture level, pH, and carbon/nitrogen ratio. It is known that nitrogen-rich materials such as poultry manure create higher temperatures in the compost pile compared to carbon-rich materials. Maintaining a compost pile is a well-established art and the various methods to control the composting process can be found in for instance The Practical Handbook of Compost Engineering—Roger T. Haug 1993, which is included herein in its entirety by reference.

When the biomass has partially completed the compost process to the necessary degree, it is removed from the system by way of a discharge port and delivered to invertebrate culture unit 2b. In smaller operations, subsystem 3b for this delivering can simply comprise a shovel, which can be used to remove the completed compost. In larger compost applications, subsystem 3b may comprise a screw auger or similar machine to transport compost from composter 1b. (see for instance The Practical Handbook of Compost Engineering—Roger T. Haug 1993, page 68, 69,72, 86). However, so as not to be introducing undesirably warm material into the invertebrate culture unit, a cooling subsystem may additionally be provided between the composter and the invertebrate culture unit in order to cool the partially composted waste in that portion of the composter to be less than about 35° C. before delivering to the invertebrate culture unit

Composter 1b includes an aeration system for maintaining aerobic conditions and an exhaust system for allowing air to leave the system. Air can be introduced into the pile by means of positive pressure, negative pressure, or a combination of the two. Piping may be distributed through the pile to deliver air to the biomass. Alternatively, the air can be allowed to passively diffuse through a pile which is well maintained to provide adequate porosity to allow gas to diffuse freely through the biomass.

An appropriate exhaust system may include pipes, ducts, or vents depending on the configuration of composter 1b. Pipes and ducts allow the air forced through the compost pile to be collected and directed to vent or can be reintroduced into the compost pile. The exhaust system may have a recycle loop to allow warm exhaust gas, still rich in moisture, to be reintroduced to the pile. Typically, make-up air is introduced into the recycled air to ensure adequate oxygen is available to the thermophylic bacteria. See for example, The Science of Composting, Part 1—Eliot Epstein 1997, page 22 or The Practical Handbook of Compost Engineering—Roger T. Haug 1993, page 261. When exhaust gas is not recycled, it is beneficial to treat the exhaust gas with a biofilter to reduce odours which are a natural byproduct of the compost product. A biofilter is not necessary for composter 1b to function but is generally recommended particularly when the compost operation is not far enough away from residences or a population center to have the odours dissipate naturally. The design and selection of biofilters for aerobic compost operations is not particularly limiting and a wide range of designs and materials are available; see for instance Odor Management, Ch 16-18 in The Practical Handbook of Compost Engineering—Roger T. Haug 1993, page 545-655.

Keeping the compost process running efficiently requires a balance to be maintained between the temperature, moisture, and pH of the pile and the level of humidity in the aeration gas. Methods of maintaining optimal process parameters are well known in the art and examples of control strategies can be found in The Practical Handbook of Compost Engineering—Roger T. Haug 1993, Process kinetics and dynamics, page 345-543. In general, the biomass should not be so moist as to allow the biomass to agglomerate and create an impermeable layer and the temperature of the biomass should be allowed to reach a temperature between 55 and 70° C. to promote the decomposition of the biomass. At these temperatures, thermophilic bacteria predominate the biotic demographic. These bacteria require a pH of between 6-8, a moisture content between 50-70%, and an air flow that keeps the bulk oxygen content in the air between 18 and 21%.

Having heat exchangers to extract heat from the composting biomass provides an additional level of control in the composting process that is not possible with conventional composting systems. With the inventive system, it is possible to reduce thermal gradients that form within the pile by circulating fluid between hotter and cooler sections of the compost pile. Such configurations are advantageous because such a system provides the ability to pre-heat new biomass added to the top of composter 1b and could also be used to thermally condition the partially composted biomass to the optimal temperature (15-25° C.) before it is introduced to invertebrate culture unit 2b.

Invertebrate culture unit 2b is used to grow macro organisms such as worms or larvae using the partially composted biomass from composter 1b in order to provide feed to vertebrates or arthropods in food culture unit 4b. While conventional composting systems are unsuitable for use in the current invention, conventional invertebrate (and particularly worm) culturing systems can be used. Any system that is designed to promote the growth and perpetuation of the worms or larvae can be used in combination with composter 1b and food culture unit 4b described here. System 20 could also combine vermicomposting and vermiculture practices in parallel or in series with each other while utilizing the same biomaterial output from composter 1b.

The solids output from composter 1b of partially composted biomass is transferred to the invertebrate culture unit 2b using suitable subsystem 3b (e.g. manually or via electric conveyer system). Invertebrate culture unit 2b represents a system in which invertebrates are raised in an environment containing partially composted biomass from which the invertebrates feed. Such invertebrates should be suitable for feeding to livestock in food culture unit 4b. Such invertebrates can include larvae, worms, and insects. Preferably invertebrate culture unit 2b is a vermiculture unit where worms break down treated biomass and produce solid worm castings.

The methods for operating a vermiculture unit are well documented in the art and the following manual provides concise methodology, equipment and useful information on building and maintaining a vermiculture system: Manual of On-Farm Vermicomposting and Vermiculture—by Glenn Munroe. The preferred worms are specific to compost environments and thrive in such conditions. The species primarily used in composting is eisenia fetida (see The Complete Technology Book on Vermiculture and Vermicompost By Niir Board, page 205).

There are many types of systems for both vermiculture and vermicomposting, and all are well described in the literature. In general, the systems provide a way of introducing biomass to the system, a method for controlling the operating conditions under which the worms exist, and a way of harvesting worms and castings from the system. A preferred system could incorporate both vermiculture and vermicompost reactors, operating in two stages; in series or in parallel. This layout could offer the benefits of both a high yield of worms and a high throughput of solids. Types of systems include windrows, bins or piles, stacked bins, and top-fed bed configurations. In vertical farming, where an emphasis is placed on maximum yield on a limited footprint, the combination of units would be preferred. Stacked bins would offer a means of vermiculture and a top-fed reactor would function as a vermicomposter, allowing the maximum worm harvest and maximum processing of organic waste.

Some of the vermiculture systems, such as stacked bins, allow for easier worm harvesting by concentrating the worms in expected locations, which are easier to separate from the bulk of the material through diverting and screening. There is some screening equipment such as the harvesters built by Jetcompost that are built specifically for this purpose.

At appropriate times, cultured feed for the vertebrates or arthropods (e.g. worms) is delivered by subsystem 5b from invertebrate culture unit 2b to food culture unit 4b. As with subsystem 3b, various convention methods and apparatus are known in the art which can be suitable for use as delivery subsystem 5b. Various vertebrates or arthropods may be grown in food culture unit 4b (e.g. chickens, fish, reptiles, amphibians, insectivorous mammals, etc.). A preferred system however can be one comprising an aquaculture unit for growing fish or other aquatic species (e.g. crustaceans). An aquaculture unit is a system where such species are raised by providing heat, food, and possibly other inputs to facilitate the growth and breeding of the livestock. Further, an aquaculture unit is a system that requires an operating temperature above ambient conditions. In the present system 20, at least some of the thermal deficit is made up by transferring heat from the composting biomass in composter 1b to food culture unit 4b. In addition, food culture unit 4b (and particularly an aquaculture unit) desirably achieves sustenance through the consumption of the macro organisms growing within invertebrate culture unit 2b.

There are many technologies and approaches to aquaculture depending on the selected species and there is ample reference literature, e.g. Aquaculture: Principles and Practices by Pillay and Kutter, 2005. The high-calorie feed that is produced by invertebrate culture unit 2b would be used to produce the feed for the aquaculture. This could be worms from vermiculture being fed to fish or crustaceans in an aquaculture unit. This feed may be mixed or processed with other feeds before being fed thereto. The general design of and operation of appropriate aqua and/or other food culture units is well known in the art and will not be described in further detail.

Excess heat from composter 1b is used to maintain optimum temperatures in the aquaculture unit by controlling the amount of heat exchange between the two. Water from the aquaculture tanks could be circulated through piping which is in communication with the biomass in composter 1b or alternatively, an independent heat exchange loop can be used to move heat from composter 1b into food culture unit (aquaculture unit) 4b.

Heat exchange subsystem 6 appears in the embodiments of FIGS. 2 and 3 in order to provide this heat exchange. In the case where system 30 is an aquaculture system (and unit 4b is an aquaculture unit), heat can be exchanged between the aquaculture tanks in unit 4b and the compost by means of a closed-loop system that runs between composter 1b and the aquaculture tanks or the water in the aquaculture tanks can be pumped through heat exchange elements in communication with the composting biomass or a combination of the two systems could be used. The latter case provides more degrees of freedom with respect to thermally conditioning the compost pile because fluids of various temperatures can be pumped through heat exchange elements located in different sections of composter 1b to optimize the temperature profile across the compost pile.

Composter 1b has located within it a function to transport heat from the exothermic compost reaction at least to food culture unit 4b in food production system 30. The heat exchange method used can be varied to suit the vessel geometry and may include plate-type heat exchangers, straight tubing or coils, or thermosyphons that may or may not be adapted to operate independent of orientation. The heat exchangers could run through or be located around the composting biomass. They could run vertically, horizontally or diagonally, within, though, or around the entire, or a portion of, the compost pile. The heat exchange elements may, or may not be, in direct contact with the biomass. The heat exchangers could be single units or could be configured as a network. It is particularly desirable to have a network of heat exchanging pipes located through the biomass because their minimal cross-sectional area prevents biomass from accumulating around the heat exchange elements.

The piping that connects the compost operation with food culture unit 4b should be insulated to avoid excessive temperature drop across the lines. The lines themselves should be compatible with the target application. The compost operation is a corrosive environment and the livestock in the animal culture unit may be sensitive to trace contaminants. A single type of tubing need not be used across the entire system. For example, corrosion-inhibited copper could be used in the compost system to take advantage of coppers heat transfer characteristics while a plastic or stainless steel tubing could be used in food culture unit 4b to eliminate the risk of copper leachate polluting the system. These examples are not meant to be limiting and are used to demonstrate the adaptability of the system.

Any fluid that can be pumped through the heat exchange system could be used to move heat from one process to the other but water is preferred as it presents no contamination risks if leaks develop either inside composter 1b or a preferred aquaculture unit 4b. The pumps used to circulate the fluid should be compatible with the application in that they should not introduce contaminants into the process stream and should be capable of overcoming the pressure drop through the piping to ensure the fluid is able to transfer the required amount of heat from the compost pile to the aquaculture unit. A standard centrifugal pump rated for water service would suffice.

A preferred embodiment is to have an insulated compost vessel of square, rectangular, or cylindrical geometry having a manifold system to distribute the aeration flow and a series of water-filled tubes running through the pile to transport heat from the compost pile to aquaculture unit 4b.

The various pumps, temperature sensors, and the like which make up heat exchange subsystem 6 are all appropriately controlled via controller 11. The control system can be automated or manual. The controller should be capable of maintaining a desirable temperature range by moderating the flow rate between systems. The controller could consist of flow restrictor valves or a pump speed controller to regulate the flow rate of the heat exchange fluid. The system could be a simple on/off control system or could be a sophisticated PID control loop as is conventionally used in the chemical process industry. The control system may also have a bypass loop which is used to decrease the temperature of the fluid before it is directed to the aquaculture unit or back to the composter.

Note that a cooling subsystem, if optionally additionally provided between the composter and the invertebrate culture unit, can serve as another additional source of heat in the heat recovery process.

### FIG. 4:

FIG. 4 shows a more detailed schematic of composter 1b to illustrate the various functional elements involved. Composter 1b comprises a vessel to contain the organic biomass, ports for loading and unloading material, 409 and 410 respectively, an aeration/exhaust system which may include input and output ducts 401, 402 respectively, for maintaining aerobic conditions throughout the compost pile, and heat exchange elements, 403 to 408 inclusive, for transferring heat from the compost pile to food culture unit 4b. Here, elements 403 and 404 represent heat exchange elements on aeration ducts 401 and 402 respectively. Elements 405 and 406 represent heat exchange elements running vertically through composter 1b. And elements 407 and 408 represent heat exchange elements running horizontally through composter 1b.

Composter 1b, including the various heat exchange elements, can be insulated to maintain high internal temperatures and/or limit the loss of heat to the surrounding environment. The heat exchange elements can be tubing, coils, plates, or thermosyphon elements. The heat exchange equipment should be filled with heat transfer fluid, preferably water, which can be circulated within composter 1b and/or pumped between composter 1b and food culture unit 4b. The heat exchange elements can have different functions depending on how the elements are configured, the source of the fluids, and in which direction the fluids flow.

Heat extracted from composter 1b is directed food culture unit 4b, but can also be directed in part to other components in a complete food production system (for instance, a hydroponic unit, an aquaponic unit, invertebrate culture unit 2b, or (through the use of a bypass loop) can be lost to the environment). The destination of the heat will be dictated by the system requirements over the course of its operation. It is also possible to direct the heat from a first composter 1b to a second such composter (not shown) thereby reducing differences in composter conditions between separate system operations.

The withdrawal of heat from composter 1b can be extensive; such as the removal of heat when the temperature of the reactor approaches the survival limit for the bacteria. Temperatures approaching 75° C. should be avoided, and therefore extracting heat to prevent harmful thermal excursions would be beneficial. Generally is it advantageous to withdraw heat from any portion of the biomass which has already reached its maximum operating temperature and has begun to cool. In this way, the residence time in the composter is minimized because the thermophilic bacteria have had maximum effect on the biomass and the resulting partially composted biomass is more quickly brought to a temperature appropriate for invertebrate culture unit 2b. Alternatively, heat can be withdrawn in an intensive fashion where heat is removed below the maximum survival temperature of the microbes. In this way, the heat exchange is creating a parasitic load on the biological combustion of the biomass and reduces the degree to which the biomass is composted.

Heat exchange elements can be in direct or indirect communication with the biomass. When in direct communication, the heat exchange elements are in physical contact with some portion of the composting biomass while indirect contact relies on heat exchange between an element in composter 1b and the heat exchange elements. Heat exchange elements located at the exterior of the housing of composter 1b, which rely on heat transferred through the composter wall, are an example of indirect contact. It is also possible to capture heat from the exhaust gases by introducing heat exchange elements to contact the warm gas stream (i.e. 404) before it exits composter 1b. It can be advantageous to maximize the surface area of contact between the warm gas and the heat exchangers by adding fins to the heat exchange elements in contact with the gas stream. Similarly, such heat exchangers can be used on the air inlet (i.e. 403) to warm the incoming air before it enters composter 1b. One or multiple heat exchange systems, in series or in parallel, may be used simultaneously to optimize the operation of system 30 and deliver heat and withdraw heat from the target locations.

It is also possible to use the heat exchange elements to break down thermal gradients that can form in the pile. Such configurations can be advantageous as they provide a uniform environment for the biomass which can reduce the time required to prepare the material for introduction into invertebrate culture unit 2b and increase the consistency of the partially digested biomass. Heat exchange elements can be configured to move heat from the high temperature portion of the pile to the lower temperature portion of the pile (i.e. 409, 410). Typically the lower temperature portion of the pile is located at the top where new biomass is introduced and thus can serve a pre-heating function which can help activate the new biomass and prepare it for composting. Similarly, it is possible to affect lateral thermal gradients by configuring the heat exchangers so that the fluid flows from the central portion of the pile to the outer portion of the pile (i.e. 407, 408).

The fluid circulating in heat exchange subsystem 6 can be fed by means of an independent fluid reservoir or can use water contained in food culture unit 4b. If multiple heat loops are in use, then one or both sources of fluid could be used. If two sources of water are being used simultaneously, then it is possible to take advantage of fluid-fluid heat exchangers, particularly counter-flow heat exchangers which are particularly efficient at exchanging heat.

### FIG. 2:

A schematic of a more comprehensive, complex exemplary food production system 20 appears in FIG. 2 that comprises the basic system 30 of FIG. 3 along with other useful subsystems. Along with the elements of the basic system 30 then, system 20 also comprises photosynthesis unit 21 and fungi unit 22 which are integrated appropriately with system 30.

Aside from the worm castings, the biomass recovered from invertebrate culture unit 2b is largely cellulose and is no longer suitable for composting. This solid material can be discarded or used as shown in prior art system of FIG. 1. Alternatively, it can be transferred by subsystem 7b to fungi unit 22 where the biomass will act as a substrate upon which edible fungi can grow. Any solids resulting from fungi unit 22 can be recycled back to composter 1b as shown by subsystem 28, or alternatively can be landfilled. Other macro organisms grown in invertebrate culture unit 2b can be used as feed in food culture unit 4b.

Screening or sieving of the completed contents of invertebrate culture unit 2b (which includes castings, worms, cocoons and undigested solids) facilitates the extraction of castings from the mixture. Once the worms and undigested solids have been separated from the treated material the treated material can be further clarified to remove worm cocoons. Screening devices for worm harvesting like the WW-Jet series by Jetcompost can also be fitted with smaller sieve sizes to separate the castings from the cocoons. The cocoons should be reintroduced with new organic material entering invertebrate culture unit 2b to maintain desirable worm yields. Alternatively, the castings can be sent to a compost tea unit (like unit 14a shown in prior art FIG. 1) to extract plant-available nutrients and microbes. The castings could also be applied directly to the base of plants, or sprinkled throughout an aquaculture tank to recycle nutrients. Further still, a liquid pesticide can be produced using the worm castings as well. This is accomplished by fermenting the castings in slurry with water in the absence of oxygen. A similar unit to that used to extract nutrients from the solid castings (not shown) can be used as long as the air input is disabled.

The liquid extract from the blend of solid castings and water within invertebrate culture unit 2b is called compost tea and it contains plant-available nutrients and healthy soil microbes. The unit that extracts the nutrients from the solid castings (not shown in FIG. 2) would steep the solid materials in oxygen rich water for a period of time sufficient to extract nutrients and microbes from the solids. This water could be city water, rainwater or be drawn from a hydroponic or an aquaculture unit of system 20 itself. There are many studies supporting the benefits of using compost tea as a nutrient solution for plants and as an addition to aquaculture. There are many manufacturers of extracters, for example Sustainable Agricultural Technologies, Inc.

Once extracted, FIG. 2 shows liquid extract from invertebrate culture unit 4b being delivered to compost tea unit 14b by subsystem 23 where it is combined with nitrified water from waste treatment unit 24. Nutrient solution from compost tea unit 14b is then delivered via subsystem 16b to photosynthesis unit 21.

Photosynthetic unit 21 utilizes nutrients to facilitate the growth of plants or other photosynthetic organisms. The organisms present in photosynthetic unit 21 should be capable of receiving digested solids from invertebrate unit 2b and/or treated waste from compost tea unit 14b and/or treated liquid waste from food culture unit 4b. Preferably photosynthetic unit 21 contains plants but may also contain photosynthetic algae, plankton, or diatoms suspended in water. Unit 21 could be open or closed (field vs. greenhouse agriculture) and, for the case of most plants, could be soil-based or soil-less (hydroponic). Preferably, photosynthetic unit 21 is a hydroponic unit where the algae or the plant roots are exposed to a nutrient-rich water stream. Different techniques offer benefits for different plant species and systems. If the hydroponic section is incorporated into an aquaponic system, then plants could be raised using a variety of hydroponic methods. An efficient method is Nutrient Film Technique which also allows photosynthesis unit 21 to be used to pull nutrients from the water. In cases where algae, plankton, or diatoms are being grown in photosynthetic unit 21, the biomass therefrom can be used to feed the livestock in food unit 4b or could go on to produce other products such as biofuel. Additionally, any solid waste from photosynthetic unit 21 can be recycled (not shown) back to composter 1b to recover energy and nutrients.

The liquid waste produced in an aquaculture system contains high levels of ammonia, which is toxic to fish. This waste stream can be discharged, or it can be treated to convert the ammonia into nitrates. Nitrification of this waste is common practice, e.g. Water and wastewater Calculations manual—2007—Dar Lin & Lee. The treated liquids can be discharged, returned to the aquaculture, can be directed to photosynthetic unit 21 to act as a fertilizer or growth promoter or can be used as a water source for making the nutrient solution from the castings from the Nutrient Recovery Unit. FIG. 2 shows liquid waste being delivered to waste treatment unit 24 by subsystem 8b whereupon it is treated and then provided to compost tea unit 14b. Solid waste from compost tea unit 14b is delivered via subsystem 29 to composter 1b.

Water is provided to photosynthesis unit 21 by subsystem 25 and to food culture unit 4b by subsystem 26. The heat produced from composter 1b may also be used to heat this water or the nutrient solution entering the unit at 16a. A nitrified water stream from aquaculture could be used as a source of water for the system. While this waste would contain some of required plant nutrients, additional nutrients could be provided by the nutrient solution stream from compost tea unit 14b. Aquaculture wastewater is less concentrated than the compost tea, therefore plant growth is limited by the concentration in the aquaculture water. Blending the two streams allows for the growth of a wider range of vegetation that has higher nutritional demands.

The liquids leaving photosynthesis unit 21 would be depleted in nutrients compared to the incoming streams. The reduced-nutrient stream could be discharged or it could be used in compost tea unit 14b to create more nutrient solution. As shown in FIG. 2 however, the nutrient reduced stream is recycled back to food culture unit 4b by subsystem 18b in a closed loop, and such an arrangement is typically called an aquaponic system. Aquaponic systems are particularly attractive configurations as they maximize the ability to recycle energy and nutrients using synergies that exist in such polyculture and even polytrophic systems.

All of the above mentioned U.S. patents and applications, foreign patents and applications and non-patent publications referred to in this specification, are incorporated herein by reference in their entirety.

While particular embodiments, aspects, and applications of the present invention have been shown and described, it is understood by those skilled in the art, that the invention is not limited thereto. Many modifications or alterations may be made by those skilled in the art without departing from the spirit and scope of the present disclosure. The invention should therefore be construed in accordance with the following claims.

What is claimed is:

1. A food production system employing waste biomass comprising:
a composter for partially composting the waste biomass;
an invertebrate culture unit operating in a temperature range selected for the invertebrate culture;
a delivery subsystem to deliver partially composted waste biomass from the composter to the invertebrate culture unit in the temperature range selected for the invertebrate culture;
a food culture unit for producing a food selected from the group consisting of vertebrates and arthropods, and the food culture unit operating in a temperature range selected for the food culture;
a delivery subsystem to deliver invertebrates from the invertebrate culture unit to the food culture unit; and
a heat exchange subsystem providing for exchange of heat from the composter to the food culture unit wherein the subsystem comprises a controller for controlling the exchange of heat such that the food culture unit is maintained in the temperature range selected for the food culture.

2. The food production system of claim 1 wherein the waste biomass is composted for less than 5 days after the time when the peak composting temperature has been reached.

3. The food production system of claim 2 wherein the waste biomass is composted for about 1 day after the time when the peak composting temperature has been reached.

4. The food production system of claim 1 wherein the invertebrate culture unit is a vermiculture unit.

5. The food production system of claim 4 wherein the temperature range selected for the vermiculture is between about 15 to 25° C.

6. The food production system of claim 1 wherein the composter is located above the invertebrate culture unit and the delivery subsystem to deliver partially composted waste is assisted by gravity.

7. The food production system of claim 6 wherein the delivery subsystem to deliver partially composted waste comprises a screw auger or a baffle.

8. The food production system of claim 1 wherein the food culture unit is an aquaculture unit.

9. The food production system of claim 8 wherein the temperature range selected for the aquaculture is above ambient temperature.

10. The food production system of claim 1 wherein the invertebrate culture unit is located above the food culture unit and the delivery subsystem to deliver invertebrates is assisted by gravity.

11. The food production system of claim 1 wherein the controller controls the temperature of at least a portion of the composter between about 50 to 70° C.

12. The food production system of claim 11 comprising a cooling subsystem between the composter and the invertebrate culture unit for cooling the partially composted waste in the portion of the composter to less than about 35° C.

13. The food production system of claim 1 wherein the heat exchange subsystem comprises a piping network and a heat exchange fluid within the piping network.

14. The food production system of claim 13 wherein the piping network is arranged throughout the composter so as to reduce any thermal gradients within the compost.

15. The food production system of claim 13 wherein the heat exchange fluid comprises water from the aquaculture unit.

16. The food production system of claim 1 comprising a photosynthesis unit coupled to the food culture unit wherein a portion of the output of the food culture unit is provided as an input to the photosynthesis unit and a portion of the output of the photosynthesis unit is provided as an input to the food culture unit.

17. The food production system of claim 1 comprising a fungi unit coupled to the composter and to the invertebrate culture unit wherein a portion of the output of the invertebrate culture unit is provided as an input to the fungi unit and a portion of the output of the fungi unit is provided as an input to the composter.

18. A method of producing food comprising:
providing a supply of waste biomass to a composter;
partially composting the waste biomass in the composter;
delivering the partially composted waste biomass to an invertebrate culture unit operating in a temperature range selected for the invertebrate culture;
culturing invertebrates in the invertebrate culture unit in the temperature range selected for the invertebrate culture;
delivering the cultured invertebrates to a food culture unit for producing a food selected from the group consisting of vertebrates and arthropods, the food culture unit operating in a temperature range selected for the food culture;
culturing food in the food culture unit;
exchanging heat from the composter to the food culture unit using a heat exchange subsystem; and
controlling the exchange of heat such that the food culture unit is maintained in the temperature range selected for the food culture.

19. The method of claim 18 comprising composting the waste biomass for less than 5 days after the time when the peak composting temperature has been reached.

20. The method of claim 18 wherein the invertebrates cultured in the invertebrate culture unit are vermi.

21. The method of claim 20 wherein the temperature range selected for the invertebrate culture is between about 15 to 25° C.

22. The method of claim 18 wherein the food cultured in the food culture unit comprises aquatic organisms.

23. The method of claim 22 wherein the temperature range selected for the food culture unit is above ambient.

24. The method of claim 18 comprising controlling the temperature of at least a portion of the composter between about 50 to 70° C.

25. The method of claim 24 comprising cooling the partially composted waste in the portion of the composter to less than about 35° C. before delivering to the invertebrate culture unit.

26. The method of claim 18 comprising using a heat exchange subsystem for the exchanging of heat wherein the subsystem comprises a piping network and a heat exchange fluid within the piping network.

27. The method of claim 26 comprising arranging the piping network throughout the composter so as to reduce any thermal gradients within the compost.

28. The method of claim 18 comprising:
providing waste solids from the invertebrate culture unit to a fungi unit;
growing fungi on the waste solids in the fungi unit; and
providing a portion of the solids from the fungi unit to the composter.

29. The method of claim 18 comprising providing waste solids from the food culture unit to the composter.

30. The method of claim 18 comprising:
preparing a nutrient solution using suspended solids and water from the food culture unit;
providing the nutrient solution to a photosynthesis unit;
growing photosynthetic organisms in the photosynthesis unit; and
providing a nutrient depleted solution from the photosynthesis unit to the food culture unit.
